(19) United States
(12) Patent Application Publication
(10) Pub. No.: US 2025/0241833 A1
(43) Pub. Date: Jul. 31, 2025

(54) SULFUR CO-GRINDING PROCESS

(71) Applicant: The Procter & Gamble Company, Cincinnati, OH (US)

(72) Inventors: Geoffrey Marc WISE, Reading, OH (US); Jose Rodel Mabilangan CARAGAY, NEWCASTLE UPON TYNE (GB)

(21) Appl. No.: 19/042,507

(22) Filed: Jan. 31, 2025

Related U.S. Application Data
(60) Provisional application No. 63/627,399, filed on Jan. 31, 2024.

(51) Int. Cl. A61K 8/23; A61K 8/368; A61K 8/41; A61Q 5/02; A61Q 5/12; A61Q 19/10

(52) U.S. Cl. CPC A61K 8/23; A61K 8/368; A61K 8/41; A61Q 5/02; A61Q 5/12; A61Q 19/10

(57) ABSTRACT

The present invention is directed to a process for making a personal care composition comprising premixing sulfur and sodium benzoate to form a blended powder.

### FIELD OF THE INVENTION

The present invention is directed to a method or process for making a personal-care composition comprising premixing sulfur and sodium benzoate to form a blended powder.

### BACKGROUND OF THE INVENTION

Sulfur is an attractive antimicrobial agent in personal-care products due to its abundance and its long history in natural medicine in many cultures. Sulfur is easily milled to a small particle size, but its strong hydrophobicity makes it difficult to disperse into water-based cosmetic formulations. Poorly dispersed sulfur creates agglomerates greater than 50 microns in the final formulation, which increase the risk of sedimentation (product instability) and consumer rejection of the product. In addition, raw sulfur is cohesive, forming clumps that do not flow.

A common method of overcoming difficulty in direct sulfur addition is to add a pre-processing step to combine sulfur with anionic or nonionic surfactants into an aqueous premix. Alternatively, a polymeric dispersant may be used, such as polynaphthalene sulfonate. However, this approach requires a structurant, such as xanthan gum, to prevent the sulfur from sedimenting out of the aqueous premix. These additional processing steps create complexity, incremental cost and risk of bacterial contamination of the aqueous premix.

The addition of materials to improve the flowability of powders has been used in the industry. Although common cheap materials, such as silica or zeolite, could be used rather than an aqueous premix, these processing aids are generally inefficient for sulfur. Moreover, these processing aids generally provide no benefit to the personal care composition, and may be objectionable to consumers. Therefore, there remains an unmet need for an efficient processing aid to enable direct addition of solid sulfur to personal-care compositions.

The present invention has surprisingly found that sodium benzoate, an ingredient commonly used as a preservative and pH buffer in personal-care formulations, can be combined with sulfur to appreciably increase its flowability and dispersibility of sulfur. This benefit is far greater than would be expected based on sodium benzoate's powder characteristics. The sodium benzoate and sulfur can be processed together in a co-milling operation, as will be evidenced in the below examples.

Moreover, the present invention has found that a sodium benzoate level as low as 10% by weight of the powder blend can be effective. Such a low level may be less than the amount of sodium benzoate that would be ordinarily added directly to the personal-care formulation, such that no additional formula cost is incurred by adding the sodium benzoate to the sulfur. The sodium benzoate can be added as the only processing aid, or in combination with another solid that is compatible with the personal-care formulation, such as tetrasodium ethylenediaminetetraacetic acid (EDTA), citric acid or sodium salicylate.

### SUMMARY OF THE INVENTION

The present invention is directed to a process for making a personal care composition comprising premixing sulfur and sodium benzoate to form a blended powder.

### DETAILED DESCRIPTION OF EMBODIMENTS OF THE INVENTION

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

While the specification concludes with claims particularly pointing out and distinctly claiming the invention, it is believed that the present invention will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “Molecular weight” refers to the weight average molecular weight unless otherwise stated. Molecular weight is measured using industry standard method, gel permeation chromatography (“GPC”).

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the hair care composition.

As used herein, “personal care compositions” includes products such as shampoos, shower gels, liquid hand cleansers, hair colorants, facial cleansers, and other surfactant-based liquid compositions

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In the present invention, successful blends of sulfur with the processing aid will result in a flowable powder blend that can be dispersed into a personal-care composition via conventional processing techniques, such as through an eductor or powder disperser. Without being bound by theory, the present invention has found that the co-milling creates a dispersion of appropriately sized sodium benzoate particles that block the sulfur particles from clumping together to reduce flowability and dispersibility. Therefore, milling parameters such as specific energy input that control the particle size distributions of sodium benzoate and sulfur in the final composition influence the flowability and dispersibility of the co-milled material. The processability of the blended powder will also depend on the weight fraction of sulfur in the blended powder composition and the material used to blend with the sulfur. For cost and formula flexibility, higher concentrations of sulfur in the powder blend may be used, such as 80%-99% by weight, 85%-98% by weight, or 90%-95% by weight sulfur in the blended powder composition. The sulfur concentration in the blended powder is thus a balance between cost and processability, since higher sulfur concentrations will tend to be more challenging to process.

In the present invention, the concentration of sulfur in the final personal-care composition can be found to meet formulation needs. In the present invention, the concentration of sulfur in the final composition may be from about 0.5% to about 5% by weight, may be from about 0.8% to about 3% by weight, may be from about 1% to about 2% by weight.

The benefit of the processing aid can be assessed via two key metrics: 1) The flowability of the combined powders, expressed as “ffc” which is the abbreviation for flow function coefficient, and the flow function of the material; and 2) The fraction of dispersed material that is below an undesirably large size, such as 50 microns.

In the present invention, ffc values may be greater than 2.0, and more than 60% of the sulfur should pass through a 50-micron sieve. Larger particle sizes of sulfur may be objectionable to consumers and may sediment out of the final product. In the present invention, the final personal-care composition may be physically stable for at least three months at 40 C, may be physically stable for at least six months at 40 C without noticeable sedimentation.

The below table illustrates the usefulness of sodium benzoate relative to other common ingredients considered as process aids. The methodologies used to generate the data on each composition are also described.

### EXAMPLES

The examples shown below demonstrate that both coblending or comilling has to be combined with the right material to be added at certain proportion in order to achieve the desired effect. Pure sulfur powder aggregates and consolidates over time resulting to a compacted powder that has lumps that do not readily flow.

The present invention may further include a dispersion of the sulfur particles in water resulting to a slurry that contain stabilizers and dispersants. This approach enables good dispersibility but has an inherent limitation of the amount of active resulting to mixtures that have relatively lower active that need to be shipped in different locations. Additionally, dispersants, stabilizers and thickeners that are used to maintain a stable dispersion are not necessarily needed in the final product.

To improve the flowability of cohesive powders, process aids are normally used. These powders typically are very fine powders (e.g. zeolite). These powders typically helps minimize the contact points between the adhesive particulates thereby improving the flow.

Surprisingly, it has been found that even a granular material such as sodium benzoate can improve the flow as long as it is co-milled in a way that deaggregates the lumps of the sulfur particles while maintaining original primary size and more importantly reducing the size of the benzoate at the same time. Addition of other materials such as EDTA and sodium salicylate enables an even better flowability as shown on its flow function while also improving its dispersibility. Given these materials are all ingredients of the desired finished product, no additional costs are invested on pure process aids. The resulting blended powder mixtures have significantly high levels of sulfur than liquid-solid dispersions, making this approach more space and cost efficient in transportation.

| | | % Passing | (24 hr Time consolidation at 6.4 | |
|---|---|---|---|---|
| | | thru 50 | kPa) | |
| | | micron sieve | Flow description | ffc |
| | | Criteria | | |
| | | >66% | cohesive flow or better | >2 |
| 100 | Pure Sulfur (as received) | 50% | No Flow | 0.55 |
| 50 | Sulfur Slurry dispersion | 66.25% | NA | NA |
| 90:10 | Sulfur-Sodium Benzoate | 93.70% | Cohesive Flow | 3.8 |
| 90:10 | Sulfur -Sodium Salicylate | 98.51% | No Flow | 0.95 |
| 90:10 | Sulfur-EDTA | 45.59% | No Flow | 0.51 |
| 85.5:9.5:5 | Sulfur-Sodium Benzoate- | 99.52% | Easy Flow | 7.88 |
| | EDTA | | | |
| 80:20 | Sulfur -Zeolite | 79.6% | Cohesive Flow | 2.1 |
| 90:10 | Sulfur -Zeolite | 81.63% | No Flow | 0.78 |
| 80:20 | Sulfur -Citric | 50.3% | No Flow | 0.58 |
| 80:20 | Sulfur -NaCl | 33.42% | Not Tested | |

Additional Data for Pure Components

Flowability

| Pure Ground | Flow | Flow |
|---|---|---|
| Materials | description | function |
| EDTA | Free Flowing | 174.80 |
| Sodium Salicylate | Very Cohesive | 1.18 |
| Sodium Benzoate | Very Cohesive | 1.21 |

In the above table, “Sulfur Slurry dispersion” is 50% sulfur by weight in water with approximately 1% polynaphthalene sulfonate (trade name Darvan 1 from Vanderbilt Minerals) as a dispersing aid. The method of measuring the ffc is described after our description of the laboratory process for generating these compositions. Details of other materials used are captured in the below table

| Material | Supplier |
|---|---|
| Sulfur | Solvay, approximately 40 micron particle size |
| Sodium Benzoate | Emerald |
| Sodium Salicylate | Alta Laboratories |
| Tetrasodium EDTA | BASF |
| Zeolite A (Sodium | PQ Europe |
| Aluminosilicate) | |
| Citric Acid | Archer Daniels Midland |
| Sodium Chloride | Morton |

### Sulfur

The personal care composition of the present invention may include sulfur. The sulfur which is suitable for use herein can be any form of elemental sulfur. Sulfur exists at room temperatures primarily as rhombic crystals. The two most prevalent ways of obtaining elemental sulfur are: precipitation from hydrogen sulfide, with one route coming from contamination in sour gas, via the Claus process and mining underground deposits using superheated water, known as the Frasch process. Other forms of sulfur, such as monoclinic crystalline sulfur, oligomeric or polymeric sulfur, are the normal primary forms which elemental sulfur assumes at certain higher temperature ranges. At room temperatures, these forms convert, or revert, to rhombic sulfur. The sulfur while being in elemental form may be sulfur which has been physically mixed with protective colloids such as gum arabic, clays, waxes, oils, activated carbon, zeolites, silica or dispersing agents such as surfactants or subjected to processing steps to modify its particle size or other physical property. Sulfur is available commercially in a variety of forms such as pellets, cakes, prills, colloidal, micronized, sublimed, precipitated, and commercial flour.

Sulfur may have a particle size distribution wherein 90% of the particles (D90) of from about 30 micron (μm) to about 250 micron (μm); Sulfur may have a particle size distribution wherein the D90 is from about 30 micron (μm) to about 200 micron (μm); Sulfur may have a particle size distribution wherein the D90 is from about 30 micron (μm) to about 150 micron (μm); Sulfur may have a particle size distribution wherein the D90 is from about 30 micron (μm) to about 100 micron (μm).

Sulfur may have a particle size distribution wherein 50% of the particles (D50) is from about 5 micron (μm) to about 150 micron (μm); Sulfur may have a particle size distribution wherein the D50 is from about 10 micron (μm) to about 100 micron (μm); Sulfur may have a particle size distribution wherein the D50 is from about 15 micron (μm) to about 75 micron (μm); Sulfur may have a particle size distribution wherein the D50 is from about 20 micron (μm) to about 50 micron (μm).

Sulfur may have a particle size distribution wherein 10% of the particles (D10) is from about 1 micron (μm) to about 25 micron (μm); Sulfur may have a particle size distribution wherein the D10 is from 5 micron (μm) to about 25 micron (μm); Sulfur may have a particle size distribution wherein the D10 is from about 10 microns (μm) to about 25 micron (μm); Sulfur may have a particle size distribution wherein the D10 is from about 18 micron (μm) to about 25 micron (μm).

Sulfur may be present in a ratio of D (90)/D (10) of from about 3 to about 100; Sulfur may be present in a ratio of D (90)/D (10) of from about 3 to about 50; Sulfur may be present in a ratio of D (90)/D (10) of from about 3 to about 10; Sulfur may be present in a ratio of D (90)/D (10) of from about 3 to about 4.

The sulfur may be present in a final composition in an amount from about 0.1% to 5%, from about 0.25% to 4%, from about 0.5% to about 3%, and from about 0.5% to 2%. The sulfur may be present in the premix in an amount from about 2% to about 60%, from about 25% to about 60%, and from about 40% to about 55%,

In the present invention it is desired to control the particle size of the sulfur after it is blended with the sodium benzoate to form the flowable powder. The desired final sulfur particle size will generally be set by the optimal size for performance in the final personal-care composition, but the impact of sulfur particle size on the powder blend's flowability should also be considered. If the applied specific energy during the blending operation is too high, then the sulfur particle size may be reduced below the target range. Conversely, if the processing temperature is too high, the sulfur particles may soften and agglomerate to an undesirably high particle size. A method to achieve this is to start with sulfur particles of the desired size, then process with sodium benzoate at a specific energy input of 1 to 100 KJ/kg at a temperature of 0 C to 70 C, such that the sulfur particle size is similar before and after the processing operation. For example, starting with a sulfur particle size of about 40 microns and obtaining a sulfur particle size of about 40 microns after the blending operation, wherein the sulfur particle size is measured with the same technique before and after the processing event. Non limiting examples of suitable sulfur particle size is described in U.S. Pat. No. 11,679,065 and US 2023/0329984A1, which are incorporated herein by reference in their entirety.

### Sodium Benzoate and Sodium Salicylate

Non limiting examples may be salicylate salts or acids, benzoate salts or acids. Non-limiting examples may be sodium salicylate, sodium benzoate, potassium salicylate, potassium benzoate, salicylic acid, benzoic acid, MEA-salicylate, MEA-benzoate, TEA-salicylate, TEA-benzoate, calcium salicylate, calcium benzoate, magnesium salicylate, magnesium benzoate, titanium salicylate, titanium benzoate, silver salicylate, silver benzoate, ammonium salicylate, ammonium benzoate, zinc salicylate, zinc benzoate, and combinations thereof.

### Detersive Surfactant

The personal care composition may comprise greater than about 10% by weight of a surfactant system which provides cleaning performance to the composition and may be greater than 12% by weight of a surfactant system which provides cleaning performance to the composition. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 25%, from about 10% to about 18%, from about 10% to about 14%, from about 10% to about 12%, from about 11% to about 20%, from about 12% to about 20%, and/or from about 12% to about 18% by weight of one or more surfactants. The pre-mix composition may comprise from about 3% to about 20%, or from about 3% to about 9% by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:
a) R₁ O(CH₂CHR₃O)_{y} SO₃M;
b) CH₃ (CH₂)_{z} CHR₂ CH₂ O (CH₂ CHR₃O)_{y} SO₃M; and
c) mixtures thereof,
where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

“Substantially free” of sulfate based surfactants as used herein means from about 0 wt % to about 3 wt %, alternatively from about 0 wt % to about 2 wt %, alternatively from about 0 wt % to about 1 wt %, alternatively from about 0 wt % to about 0.5 wt %, alternatively from about 0 wt % to about 0.25 wt %, alternatively from about 0 wt % to about 0.1 wt %, alternatively from about 0 wt % to about 0.05 wt %, alternatively from about 0 wt % to about 0.01 wt %, alternatively from about 0 wt % to about 0.001 wt %, and/or alternatively free of sulfates. As used herein, “free of” means 0 wt %.

The hair care composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The hair care composition may further comprise from about 0.25% to about 15%, from about 1% to about 14%, from about 2% to about 13% by weight of one or more amphoteric, zwitterionic, nonionic co-surfactants, or a mixture thereof.

Suitable amphoteric or zwitterionic surfactants for use in the hair care composition herein include those which are known for use in shampoo or other hair care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, those selected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium disodium capryloamphodiacetate, capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidocthyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaincamido amphopropionate, coco-betaine, coco-hydroxysultaine, coco/olcamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Olcamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Texas under the trade names Neodol® 91, Neodol® 23, Neodol® 25, Neodol® 45, Neodol® 135, Neodo®l 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Delaware, including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tx.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tx.) and octylphenol ethoxylates (Triton™ X-15, X-35, X-45, X-114, X-100, X-102, X-165, X-305, X-405, X-705 available from The Dow Chemical Company, Houston, TX).

Also suitable for use herein are tertiary alkylamine oxides including lauramine oxide and cocamine oxide.

Non limiting examples of other anionic, zwitterionic, amphoteric, and non-ionic additional surfactants suitable for use in the hair care composition are described in Mccutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, alternatively from about 1% to about 25%, alternatively from about 2% to about 20%. The surfactant combination can have a cumulative average weight % of C8 to C12 alkyl chain lengths of from about 7.5% to about 25%, alternatively from about 10% to about 22.5%, alternatively from about 10% to about 20%. The surfactant combination can have an average C8-C12/C13-C18 alkyl chain ratio from about 3 to about 200, alternatively from about 25 to about 175.5, alternatively from about 50 to about 150, alternatively from about 75 to about 125.

### Additional Components

The personal care composition described herein may be a shampoo, conditioner, and/or leave-on treatments and may optionally comprise one or more additional components known for use in hair care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the personal care compositions.

Non-limiting examples of additional components for use in the personal care compositions include conditioning agents, natural cationic deposition polymers, synthetic cationic deposition polymers, anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

### 1. Conditioning Agent

The personal care compositions may comprise one or more conditioning agents. Conditioning agents include materials that are used to give a particular conditioning benefit to hair. The conditioning agents useful in the hair care compositions of the present invention typically comprise a water-insoluble, water-dispersible, non-volatile, liquid that forms emulsified, liquid particles. Suitable conditioning agents for use in the hair care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt % to about 10 wt %, from about 0.1 wt % to about 8 wt %, and from about 0.2 wt % to about 4 wt %, by weight of the composition.

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

### Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

### Benefit Agents

The personal care composition may further comprise one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, perfumes, brighteners, enzymes, sensates, attractants, dyes, pigments, bleaches, and mixtures thereof. In the present invention, the personal care composition may further comprise one or more optional ingredients, including scalp health agents and benefit agents. Suitable scalp health agents and benefit agents include, but are not limited to conditioning agents, cationic polymers, silicone emulsions, anti-dandruff agents such as polyvalent metal salts of pyrithione, non-limiting examples include zinc pyrithione (ZPT) and copper pyrithione, or selenium sulfide, benefit agents providing moisturization, barrier improvement, anti-fungal, anti-microbial and anti-oxidant, anti-itch, and sensates, gel networks, chelating agents, and natural oils such as sun flower oil or castor oil. Additional suitable optional ingredients include but are not limited to perfumes, perfume microcapsules, colorants, particles, anti-microbials, foam busters, anti-static agents, rheology modifiers and thickeners, emulsifiers, suspension materials and structurants, pH adjusting agents and buffers, preservatives, pearlescent agents, solvents, diluents, anti-oxidants, vitamins, vitamin E and F, salicylic acid, niacinamide, caffeine, panthenol, zinc oxide, zinc carbonate, basic zinc carbonate, glycols, glycolic acid, PCA, PEGs, erythritol, glycerin, triclosan, lactates, hyaluronates, allantoin and other ureas, betaines, sorbitol, glutamates, xylitols, menthol, menthyl lactate, iso cyclomone, benzyl alcohol, coal tar, charcoal, whitfield's ointment, castellani's paint, aluminum chloride, gentian violet, octopirox (piroctone olamine), ciclopirox olamine, undecylenic acid and its metal salts, azoxystrobin and other strobulins, potassium permanganate, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, itraconazole, ketoconazole benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, terconazole and combinations thereof. In the present invention, a perfume may be present from about 0.5% to about 7%.

One or more stabilizers can be included. For example, one or more of ethylene glycol distearate, citric, citrate, a preservative such as kathon, sodium chloride, sodium benzoate, and ethylenediaminetetraacetic acid (“EDTA”) can be included to improve the lifespan of a personal care composition.

The personal care compositions of the present invention may be presented in typical personal care formulations. They may be in the form of solutions, dispersion, emulsions, powders, tales, encapsulated, spheres, spongers, solid dosage forms, foams, and other delivery mechanisms. The compositions of the present invention may be hair tonics, leave-on hair products such as treatment, and styling products, rinse-off hair products such as shampoos, and treatment products; and any other form that may be applied to hair.

The personal care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The personal care composition may be in a single phase or a single product, or may be in a separate phases or separate products. If two products are used, the products may be used together, at the same time or sequentially. Sequential use may occur in a short period of time, such as immediately after the use of one product, or it may occur over a period of hours or days.

### Powder Blending Processes

### Laboratory Scale Batch Process Description

A batch of 500-gram premix is prepared by first pre-weighing the appropriate weights of each component of the composition. The materials are then transferred to a mixer (Kenwood FDP 30 food mixer). Kenwood FDP 30 food mixer is a non-limiting example and other conventional mixers may be used. The mixer is switched on to set point 1 for 2 minutes to allow the powder to mix while ensuring minimal dust leakage and formation. Once the mixing is complete, the mixture is divided into five 100-gram samples. Each sample is loaded into a bench top grinder (Delonghi KG49 Coffee grinder) and subjected to five 5-second grinding intervals in the bench top grinder. Delonghi KG49 Coffee grinder is a non-limiting example and other conventional grinders may be used. This grinding process is repeated for each 100-gram sample until all 500 grams have been similarly processed. For pure sulfur, the initial mixing step is omitted but identical grinding step is followed.

### Laboratory Scale Continuous Process

A Netzsch CUM 150 pin mill grinder with a single volumetric feeder is set up. Netzsch CUM 150 pin mill grinder is a non-limiting example and other conventional grinders may be used. Sulfur and Sodium Benzoate powders are first pre weighed as per the required composition. The material is passed through a double screw feeder for additional mixing and delumping of large particles. The mixture passes through a funnel connected to a rotary valve before entering the inlet of the pin mill grinder. Nitrogen gas is added in between the rotary valve and inlet grinder to inert the system and recycled back after passing through the grinder. Pressure indicator is installed in between the rotary valve and grinder inlet while oxygen content indicator is added after the grinder outlet. Nitrogen is first added at the correct flowrate into the system to maintain oxygen levels below the Limiting Oxygen Content (LOC) as dictated by the material property. Once LOC is reached, the grinder is turned on and Nitrogen flowrate is adjusted accordingly to maintain below LOC. As powder is added via the feeders, oxygen is monitored and maintained consistently below the LOC. Excess gas exits the filter sock installed on the unit. A dust extraction arm is placed on top of the filter sock. The ground material is collected after the grinder or after another rotary valve placed after the grinder.

### Pilot Scale Continuous Process

A 90:10 sulfur: benzoate and 85.5:9.5:5 sulfur:benzoate:EDTA compositions are also made using a RETSCH™ pin mill at a total solids feed rate of 1 kg/min and specific energy inputs of 1-100 KJ/kg, where the specific energy input is calculated by dividing the mill's power input by the powder mass feed rate. A RETSCH™ pin mill is a non-limiting example and other conventional mills may be used. For both the 90:10 and 85.5:9.5:5 blended powders, 4.5 kg is successfully incorporated into a 200-kg batch of a personal care composition using a Quadro Y-tron ZC0 powder disperser. Quadro Y-tron ZC0 powder disperser is a non-limiting example and other conventional dispersers may be used.

The present invention may incorporate the blended powder into the personal care composition using an eductor or powder dispersing device.

### Test Methods

### Flowability Test: Uniaxial 24-Hour Consolidation

This test measures the relative flowability of the powder and its corresponding mixtures using uniaxial consolidation test and calculating the resulting unconfined yield strength of the powder.

The uniaxial cake test unit comprises of the following: 1) A cylinder made up of a solid plastic with polished surface having a diameter of 5.9 cm, a length of 17.7 cm and a base plate to support the cylinder on one end. 6 slots are placed through the cylinder to allow adjustment of the initial powder volume. 2) A hollow plastic cylindrical sleeve with polished inner surface with inner diameter of approximately 6 cm to fit closely with the cylinder with a length of 16.5 cm and wall thickness of 0.5 cm. 3) A plastic lid that is a disc to fit over the sleeve and enough to support the weights to be used. 4) A Texture Analyzer (Stable micro systems and TA.XT plus) with a load cell of 30 kg, force sensitivity of 0.5 grams and a movable arm set at 10.2 mm/min. A flat circular disc plate with a diameter larger than the cylindrical sleeve is attached to the arm to be used to break the formed cake.

The cylinder is placed on a flat surface and may be on a removable tray that can catch the powder after the test. The sleeve is then placed on the cylinder supported by the pin set at a pre-determined level appropriate for the powder. This level is considered acceptable if the resulting aspect ratio of the consolidated powder is within 1.2 to 1.4 whereby the aspect ratio is defined as the ratio between the bed height of the consolidated powder divided by the diameter of the sleeve.

The powder to be tested is then poured over the empty cylindrical volume formed by the sleeve and the cylinder until the material overflows on the sides. Any vibration must be avoided to prevent the powder from consolidating while the cylinder is being filled. Level off the powder by removing excess powder using a straight edge rule or spatula. The lid is then placed on centre top of the sleeve. A 1.85 kg metal weight is then placed on top of the lid. The metal pin is carefully removed to allow the weight to consolidate the powder. This is set aside for 24 hours. After the 24-hour consolidation is completed, the weight is carefully removed. The setup is placed on the Texture Analyzer (TA) ensuring the set up remains intact. The lid is removed in a rotating and lifting motion. The sleeve is pulled slowly downwards in a rotating motion to fully expose the formed cake.

The TA arm with the metal flat disc is then positioned 5 cm above the cake. The test is programmed for compression mode allowing the arm to travel at the set speed of 10.2 mm/min with 5 g trigger force and distance of 20 mm. Force-distance is generated during the test while the maximum force in kg is recorded to break the cake in such a way that the break line intersects the left and side walls of the formed cake.

The unconfined yield strength (ffc) is then calculated using the following equation:

$$ffc = Stress due to compaction ( Pa ) = weight used to form the cake , kgf \\ Maximum Stress to break the formed cake ( Pa ) \\ Maximum Force to break the cake , kgf$$

The resulting ffc is then classified accordingly to the following descriptors.
ffc<1: No flow—unacceptable
1<ffc<2: Very cohesive—unacceptable
2<ffc<4: Cohesive—acceptable
4<ffc<10: Easy flowing—acceptable
10<ffc: free flowing—acceptable

Powders and Bulk Solids—Behavior, Characterization, Storage and Flow by Dietmar Schulze 2nd ed. 2021, published by Springer (Link: https://www.springer.com/us/book/9783030767198) incorporated herein by reference.

Other potential methods of achieving benefits of Sodium Benzoate
Premixing of ground Sulfur and ground sodium benzoate
Inline co-grinding of Sulfur Benzoate mixtures
Achieving specific polydispersity of combined Sulfur-Benzoate (span of at least 2)

### Dispersibility Test

This test measures the dispersibility of the powder composition in a personal-care base to form a personal care composition, such as a shampoo, conditioner or body wash. To avoid product instability and consumer noticeability of agglomerated sulfur in use, it is generally desired to have at least 60%, may be at least 90% of the mass of sulfur in the final personal care composition be less than 50 microns. The test requires the following items: 200 ml beaker; Overhead agitator with pitched-blade Impeller; 50-micron stainless steel sieve, 9 cm diameter; Vacuum filtration set up consisting of the following: Vacuum pump (model number XX), 5L Flask, Buchner funnel, Hartley pattern without the perforated disk, Air tubing, clips and Rubber conical sleeves.

### Personal Care Base

### Example Personal Care Base Composition

| Raw material | Supplier | Grams |
|---|---|---|
| 25% sodium laureth-1 sulfate | Stepan | 30.66 |
| Jaguar c500 guar hydyroxypropyltrimonium | Solvay | 0.19 |
| chloride | | |
| 85% Coconut monoethanolamide | Stepan | 0.76 |
| 30% Cocamidopropyl betaine | Evonik | 3.23 |
| Sodium benzoate | Emerald | 0.17 |
| Citric acid | Archer Daniels Midland | 0.21 |
| 1.5% Kathon CG (preservative) | Dow Chemical | 0.022 |
| Deionized water | — | 24.76 |

60 grams of base is pre-weighed in a 200 mL beaker. The powder to be tested is weighed separately, wherein the amount of pure sulfur in the test powder is 1.2 g. For example, for a powder than is 90% sulfur and 10% processing aid, the amount of test powder would be 1.2 g/90%=1.33 g.

The powder blend is added to the base and mixed via impeller for ten minutes to disperse the sulfur and dissolve the water-soluble component of the powder blend. The resulting mixture is then equally divided into 3 vials of 20 grams each.

The 50-micron stainless steel sieve is first pre-weighed, and the weight is recorded. It is placed in between the top cylinder and the Buchner funnel supported by metal clips to keep it in place. This is then placed on top of the 5 L flask with the rubber conical sleeves in between to have a good seal. The flask is connected to a vacuum pump. After turning on the vacuum pump, the 20-gram mixture is slowly poured over the exposed area of the sieve. The undissolved, undispersed and Sulfur particles larger than 50-micron are retained on the metal sieve. 1L of demineralized water is poured over the undissolved particles on the sieve to rinse off the base and remove any small Sulfur particles weakly aggregated or stuck on the sieve. Then may use part of the 1L demineralized water to rinse the vial and get all the samples out of it. Then the vacuum is continued for additional 2 minutes to remove excess water. The sieve with the sample is removed and then dried in an 80 C oven for 1 hr. Once the samples are dried, the weight is recorded, and the difference between the dried sample and the empty sieve is calculated. The weight of dried Sulfur sample1 is recorded. The steps are then repeated as described above for the remaining 2 samples in the vials. Then the weights of the sample are added and divided by the weight of the pure Sulfur (1.2 g) get the % retained on 50 micron. The % Sulfur passing through 50 micron is calculated by subtracting the % retained Sulfur on 50 micron from 1 as shown below.

$$Wt of dried Sulfur sample 1 = Weight of sieve with dried sulfur - weight of empty sieve \\ Total weight of sample after drying = Sum of Wt of dried Sulfur sample 1 , 2 and 3 \\ \% retained on 50 micron - total weight of sample after drying / initial weight of pure Sulfur \times 100 \\ \% passing through 50 - micron = 100 \% minus the \% retained on 50 - micron$$

### Additional Personal Care Examples

The blended powder of the present invention is suitable for inclusion in a wide range of personal care compositions. Below is a non-limiting table of example personal care compositions that incorporate the present invention. All material levels in the table are on a 100% active basis. For these compositions, the added sodium benzoate is the sum of directly added sodium benzoate and sodium benzoate delivered through the use of the blended powder. Examples A-D, F, and H-J are non-limiting examples of a shampoo composition. Example E is a non-limiting example of a conditioner composition. Example G is a non-limiting example of a body wash composition.

| Formula Examples | A | B | C | D | E | F | G | H | I | J |
|---|---|---|---|---|---|---|---|---|---|---|
| Sodium Laureth-1 Sulfate¹ | 13 | 0 | 0 | 13 | 0 | 12.5 | 0 | 0 | 0 | 0 |
| Sodium Lauryl Sulfate² | 0 | 6 | 8 | 0 | 0 | 1.5 | 6.7 | 0 | 0 | 13 |
| Sodium Laureth-3 Sulfate³ | 0 | 6 | 0 | 0 | 0 | 0 | 0 | 0 | 0 | 0 |
| Alpha Olefin Sulfonate⁴ | 0 | 0 | 4 | 0 | 0 | 0 | 0 | 12 | 0 | 0 |
| Sodium Trideceth-2 | 0 | 0 | 0 | 0 | 0 | 0 | 6.6 | 0 | 0 | 0 |
| sulfate⁵ | | | | | | | | | | |
| Sodium Cocoyl | 0 | 0 | 0 | 0 | 0 | 0 | 0 | 1.5 | 6.0 | 0 |
| Isethionate⁶ | | | | | | | | | | |
| Cocamidopropyl Betaine⁷ | 2.4 | 1.6 | 2.4 | 3.5 | 0 | 1.2 | 6.7 | 4.2 | 0 | 2.4 |
| Lauramidopropyl Betaine⁸ | 0 | 0 | 0 | 0 | 0 | 0 | 0 | 0 | 8 | 0 |
| Cocamide MEA⁹ | 0 | 0.9 | 0 | 0 | 0 | 1 | 0 | 0 | 0 | 0 |
| Stearylamidopropyl | 0 | 0 | 0 | 0 | 1.2 | 0 | 0 | 0 | 0 | 0 |
| dimethylamine¹⁰ | | | | | | | | | | |
| Trideceth-3¹¹ | 0 | 0 | 0 | 0 | 0 | 0 | 1.6 | 0 | 0 | 0 |
| Hydroxyethylcellulose¹² | 0 | 0 | 0 | 0.05 | 0.4 | 0 | 1 | 0 | 0 | 0 |
| Polyquaternium- | 0.12 | 0 | 0 | 0.05 | 0 | 0 | 0 | 0 | 0 | .08 |
| 6(DADMAC)¹³ | | | | | | | | | | |
| Guar | 0.3 | 0.25 | 0.35 | 0.35 | 0 | 0.3 | 0.4 | 0 | 0 | .25 |
| Hydroxypropyltrimonium | | | | | | | | | | |
| Chloride¹⁴ | | | | | | | | | | |
| Polyquaternium-76¹⁵ | 0 | 0 | 0 | 0 | 0 | 0.1 | 0 | 0 | 0 | 0 |
| Dimethicone¹⁶ | 2 | 1 | 2 | 0.5 | 1 | 2.2 | 0 | 1.5 | 0 | 0 |
| Sulfur¹⁷ | 2 | 2 | 1 | 1 | 0.3 | 3 | 2 | 1 | 1 | 5 |
| Piroctone Olamine¹⁸ | 0 | 0 | 0 | 0 | 0 | 0.5 | 0 | 0 | 0 | 0.5 |
| Salicylic Acid¹⁹ | 0 | 2 | 0 | 0 | 0 | 0 | 0 | 0 | 0 | 0 |
| Benzyl Alcohol²⁰ | 0 | 0.5 | 0 | 0 | 0.5 | 0 | 0 | 0 | 0 | 0 |
| Sodium Citrate²¹ | 0 | 0 | 0 | 0 | 0 | 0 | 0 | 0.78 | 0 | 0 |
| Sodium Chloride²² | 0.0 | 2.4 | 0.5 | 0 | 0 | 1.6 | 4.7 | 0.4 | 0 | 0 |
| Sodium Xylene Sulfonate²³ | 1.1 | 0 | 0 | 0.5 | 0 | 0 | 0 | 0 | 0 | 1 |
| Sodium Salicylate²⁴ | 0 | 0 | 0 | 0 | 0 | 0 | 0 | 0.45 | 0 | .25 |
| Sodium Benzoate²⁵ | 0.25 | 0.25 | 0.25 | 0.25 | 0.1 | 0.45 | 0.35 | 0.8 | 0.45 | .25 |
| Methylchloroisothiazolinone/ | 0.0005 | 0.0005 | 0.0005 | 0.0005 | 0.0005 | 0.0005 | 0.0005 | 0 | 0 | 0 |
| Methylisothiazolinone²⁶ | | | | | | | | | | |
| Citric Acid²⁷ | 0.3 | 0.5 | 0.3 | 0.3 | 0.2 | 0.4 | 0.4 | 0.5 | 0.3 | .25 |
| Glycol Distearate²⁸ | 2.5 | 2.5 | 2.4 | 2.8 | 0 | 1.7 | 3.0 | 3.0 | 3.0 | 0 |
| Acrylates Copolymer²⁹ | 0.5 | 0.3 | 0.2 | 0.2 | 0 | 0 | 0.3 | 0 | 0 | 0 |
| Trihydroxystearin³⁰ | 0 | 0 | 0.1 | 0 | 0.0 | 0.3 | 0 | 0 | 0 | 0 |
| Stearyl Alcohol³¹ | 0.2 | 0 | 0.64 | 0.64 | 1.4 | 0 | 0 | 0 | 0 | 0 |
| Cetyl Alcohol³² | 0.4 | 0 | 0.32 | 0.32 | 1.0 | 0 | 0 | 0 | 0 | 0 |
| Dipropylene Glycol³³ | 0 | 0 | 0 | 0 | 0 | 0 | 3 | 0 | 0 | 0 |
| Glycerin³⁴ | 0 | 2 | 0 | 0 | 0.3 | 0 | 0 | 0 | 0 | 0 |
| Fragrance | 0.9 | 1.2 | 1 | 1 | 0.5 | 1 | 1 | 1.2 | 1.0 | 1.0 |
| Water , QS to 100 | qs | qs | qs | qs | qs | qs | qs | qs | qs | qs |

Ingredients legend:
¹Sodium Laureth-1 Sulfate at 26% active, supplier: Procter & Gamble (P&G)
²Sodium Lauryl Sulfate at 29% active, supplier: P&G
³Sodium Alkyl Ether Sulfate, 28% active, supplier P&G
⁴Bio-Terge AS40, 40% active, supplier: Stepan
⁵Sodium Trideceth-2 Sulfate, 45% active, supplier P&G
⁶HOSTAPON SCI 85 C, supplier Clariant
⁷Tego Betain L 7 OK at 30% active, supplier: Evonik
⁸Lauramidopropyl Betaine Mackam DAB ULS, 35% active, supplier: Solvay
⁹Ninol Comf at 85% active, supplier: Stepan
¹⁰Incromine SD, supplier: Croda
¹¹Alkosynt IT30, supplier Oxiteno
¹²Natrasol 250 HHX, supplier Ashland
¹³Flocare C106, supplier: SNF
¹⁴N-HANCE 3196 Guar Gum, supplier: Ashland
¹⁵Mirapol 100 at 31.5% active, supplier: SNF, Inc.
¹⁶CF330M, supplier: Momentive
¹⁷Sulfur, supplier: Solvay
¹⁸Octopirox, supplier: Clariant
¹⁹Salicylic acid, supplier: Salicylates and Chemicals
²⁰Benzyl Alcohol, supplier: Valtris
²¹Sodium Citrate Dihydrate, supplier: ADM
²²Sodium Chloride, supplier: Morton; level adjustable to control viscosity
²³Stepanate SXS, supplier: Stepan; level adjustable to control viscosity
²⁴Sodium Salicylate, supplier: Spectrum Chemical
²⁵Sodium Benzoate Dense NF/FCC, supplier: Emerald Performance Materials
²⁶Kathon CG at 1.5% active, supplier: Dow
²⁷Citric Acid Anhydrous, supplier: ADM; level adjustable to achieve target pH
²⁸TEGIN G 1100, supplier: Evonik
²⁹Carbopol Aqua SF-1 at 30% active, supplier: Lubrizol
³⁰Thixcin R, supplier: Elementis
³¹Stearyl Alcohol, supplier: P&G Chemicals
³²Cetyl Alcohol, supplier: P&G Chemicals
³³Dipropylene Glycol, supplier: BASF
³⁴Glycerin, supplier: P&G

### ADDITIONAL EXAMPLES/COMBINATIONS

A. A process for making a personal care composition comprising premixing sulfur and sodium benzoate to form a blended powder.
B. A process for making a personal care composition according to Paragraph A, wherein the blended powder is at least 80% sulfur by weight.
C. A process for making a personal care composition according to Paragraph A-B, wherein the blended power is a ratio of 90:10 of sulfur:sodium benzoate.
D. A process for making a personal care composition according to Paragraph A-C, wherein the blended powder further comprises a material selected from the group consisting of tetrasodium ethylenediaminetetraacetic acid (EDTA), citric acid, sodium salicylate and mixtures thereof.
E. A process for making a personal care composition according to Paragraph A-D, wherein the blended powder is a ratio of 85.5:9.5:5 of sulfur:sodium benzoate:tetrasodium ethylenediaminetetraacetic acid (EDTA).
F. A process for making a personal care composition according to Paragraph A-E, wherein a specific energy input during a powder blending operation is between 1 and 100 KJ/kg.
G. A process for making a personal care composition according to Paragraph A-F, wherein a flow function coefficient (ffc) of the blended powder is greater than 2.
H. A process for making a personal care composition according to Paragraph A-G, wherein a flow function coefficient (ffc) of the blended powder is between 4 and 10.
I. A process for making a personal care composition according to Paragraph A-H, wherein more than 60% of the sulfur in the blended powder in the personal care composition passes through a 50-micron sieve.
J. A process for making a personal care composition according to Paragraph A-I, wherein more than 90% of the sulfur in the blended powder in the personal care composition passes through a 50-micron sieve.
K. A process for making a personal care composition according to Paragraph A-J, wherein the blended powder is incorporated into the personal care composition using an eductor or powder dispersing device.
L. A process for making a personal care composition according to Paragraph A-K, wherein the personal care composition contains between 0.5% and 5% sulfur.
M. A process for making a personal care composition according to Paragraph A-L, wherein the personal care composition is stable for at least three months at 40° C.
N. A process for making a personal care composition according to Paragraph A-M, wherein the personal care composition is stable for at least three months at 40° C. without noticeable sedimentation.
O. A process for making a personal care composition according to Paragraph A-N, wherein the personal care composition is selected from the group consisting of a shampoo, a conditioner or a body wash.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A process for making a personal care composition comprising premixing sulfur and sodium benzoate to form a blended powder.

2. The process for making a personal care composition according to claim 1, wherein the blended powder is at least 80% sulfur by weight.

3. The process for making a personal care composition according to claim 1, wherein the blended power is a ratio of 90:10 of sulfur:sodium benzoate.

4. The process for making a personal care composition according to claim 1, wherein the blended powder further comprises a material selected from the group consisting of tetrasodium ethylenediaminetetraacetic acid (EDTA), citric acid, sodium salicylate and mixtures thereof.

5. The process for making a personal care composition according to claim 4, wherein the blended powder is a ratio of 85.5:9.5:5 of sulfur:sodium benzoate:tetrasodium ethylenediaminetetraacetic acid (EDTA).

6. The process for making a personal care composition according to claim 1, wherein a specific energy input during a powder blending operation is between 1 and 100 KJ/kg.

7. The process for making a personal care composition according to claim 1, wherein a flow function coefficient (ffc) of the blended powder is greater than 2.

8. The process for making a personal care composition according to claim 1, wherein a flow function coefficient (ffc) of the blended powder is between 4 and 10.

9. The process for making a personal care composition according to claim 1, wherein more than 60% of the sulfur in the blended powder in the personal care composition passes through a 50-micron sieve.

10. The process for making a personal care composition according to claim 7, wherein more than 90% of the sulfur in the blended powder in the personal care composition passes through a 50-micron sieve.

11. The process for making a personal care composition according to claim 1, wherein the blended powder is incorporated into the personal care composition using an eductor or powder dispersing device.

12. The process for making a personal care composition according to claim 1, wherein the personal care composition contains between 0.5% and 5% sulfur.

13. The process for making a personal care composition according to claim 1, wherein the personal care composition is stable for at least three months at 40° C.

14. The process for making a personal care composition according to claim 13, wherein the personal care composition is stable for at least three months at 40° C. without noticeable sedimentation.

15. The process for making a personal care composition according to claim 1, wherein the personal care composition is selected from the group consisting of a shampoo, a conditioner or a body wash.
